(19) United States
(12) Plant Patent Application Publication
(10) Pub. No.: US 2003/0177551 P1
(43) Pub. Date: Sep. 18, 2003

(54) Hybrid tea rose plant named 'KORpries'

(75) Inventor: Tim-Hermann Kordes, Offenseth-Sparrieshoop (DE)

Correspondence Address: Russell D. Orkin, 700 Koppers Building, 436 Seventh Avenue, Pittsburgh, PA, 15219-1818, US

(73) Assignee: W. Kordes Sohne Rosenschulen GMBH & Co. KG

(21) Appl. No.: 10/100,436

(22) Filed: Mar. 18, 2002

(51) Int. Cl. A01H 5/00

(52) U.S. Cl. PLT/137

(57) ABSTRACT

This invention relates to a new and distinct variety of hybrid tea rose plant named ‘KORpries’ which has attractive pink colored flowers and attractive dark green foliage. This new variety is characterized by having a high production of long stems with a long vase life, vigorous and upright growth, and good tolerance to powdery mildew. The variety successfully propagates from softwood cuttings and is very suitable for year round production of cut flowers in commercial glass houses as a flowering cut rose.

### BACKGROUND OF THE INVENTION

The present invention constitutes a new and distinct variety of hybrid tea rose plant named ‘KORpries’ which was developed by artificially pollinating an unnamed, unpatented seedling with an unnamed, unpatented seedling. The two parents were crossed in the summer of 1999 and the resulting seed was sown in December 1999 in a controlled greenhouse environment. The objectives of the hybridization was to create a new and distinct rose variety for commercial greenhouse with the following characteristics:

1. Uniform, large buds that are well turbinated and flowers with good vase-life.

2. Novel flower color.

3. High cut flower production, with medium to long stems.

4. Vigorous, upright growth.

5. Suitability for production from softwood cuttings.

6. Good tolerance to powdery mildew.

7. Suitability for production of cut flowers under greenhouse growing conditions.

This combination of qualities was not present in previously available commercial cultivars of this type and distinguish ‘KORpries’ from other varieties known to the inventor.

The seeds from hybridization were planted in a controlled environment and evaluations were conducted on the resulting plants. ‘KORpries’ was selected in a development program in Klein Offenseth, Sparrieshoop, Germany.

Asexual reproduction of ‘KORpries’ by cuttings was first done in Germany and later in California. The reproductions are done by cuttings in controlled greenhouse environments and have confirmed that the distinguishing features of the new cultivar come true, remain stable, and are retained through successive propagations. ‘KORpries’ is a highly productive hybrid tea rose with a vase life greater than 11 days. Under conventional greenhouse conditions in Germany (64° C. nights, 26° C. days), ‘KORpries’ will produce 220 stems averaging 55 cm to 60 cm long.

The new rose may be distinguished from its seed parent by the following combination of characteristics:

1. ‘KORpries’ has big double flowers, while the unnamed seedling has big single flowers.

2. ‘KORpries’ has pink colored petals, while the unnamed seedling has dark orange-red petals.

The new variety may be distinguished from its pollen parent by the following combination of characteristics:

1. ‘KORpries’ has smaller flowers and foliage as compared to the unnamed seedling.

2. ‘KORpries’ has pink colored petals, while the unnamed seedling has cream-white petals.

### DESCRIPTION OF THE DRAWINGS

The accompanying color illustrations show as true as possible in color photographs of this type, the typical characteristics of the buds, flowers, leaves, stems of ‘KORpries’. Specifically illustrated as follows:

In Photo sheet #1:

FIG. 1 illustrates a young shoot;

FIG. 2 illustrates a bud before opening of the sepals;

FIG. 3 illustrates a bud at the opening of the sepals;

FIG. 4 illustrates a bud at the opening of the petals;

FIG. 5 illustrates a flower during the course of opening;

FIG. 6 illustrates an open flower, plan obverse view;

FIG. 7 illustrates an open flower, plan reverse view;

FIG. 8 illustrates a fully open flower, plan obverse view; and

FIG. 9 illustrates a fully open flower, plan reverse view.

In Photo sheet #2:

FIG. 10 illustrates a receptacle showing stamens and pistils;

FIG. 11 illustrates a receptacle showing pistils (stamens removed);

FIG. 12 illustrates flower petals, detached, outer surface;

FIG. 13 illustrates flower petals, detached, inner surface;

FIG. 14 illustrates a bare stem exhibiting thorns and flower attachment;

FIG. 15 illustrates 3 leaflets, upper side;

FIG. 16 illustrates 3 leaflets, under side;

FIG. 17 illustrates 5 leaflets, upper side; and

FIG. 18 illustrates 5 leaflets, under side.

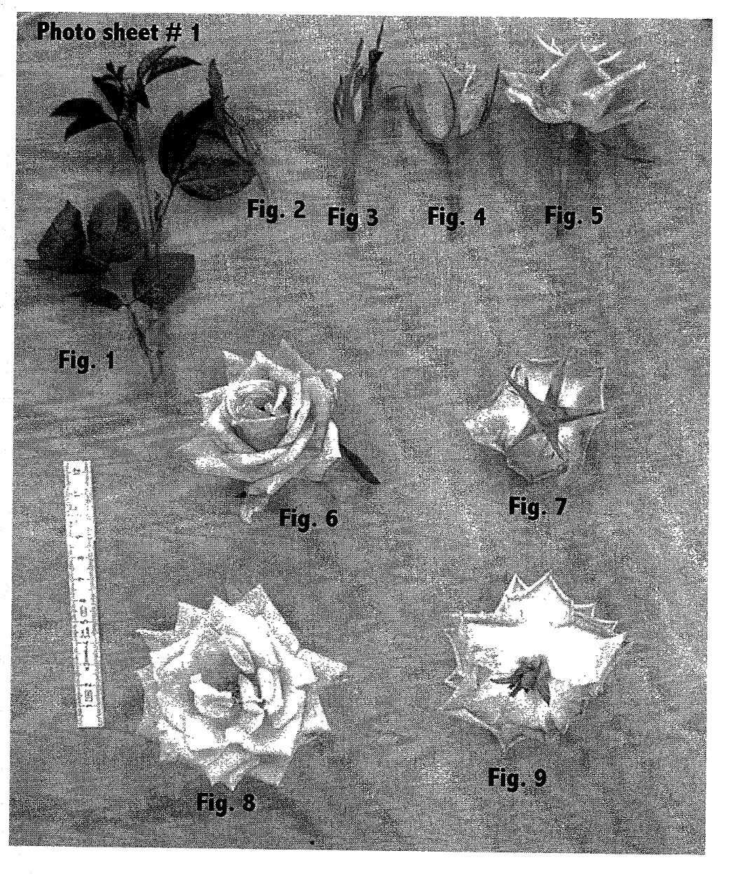

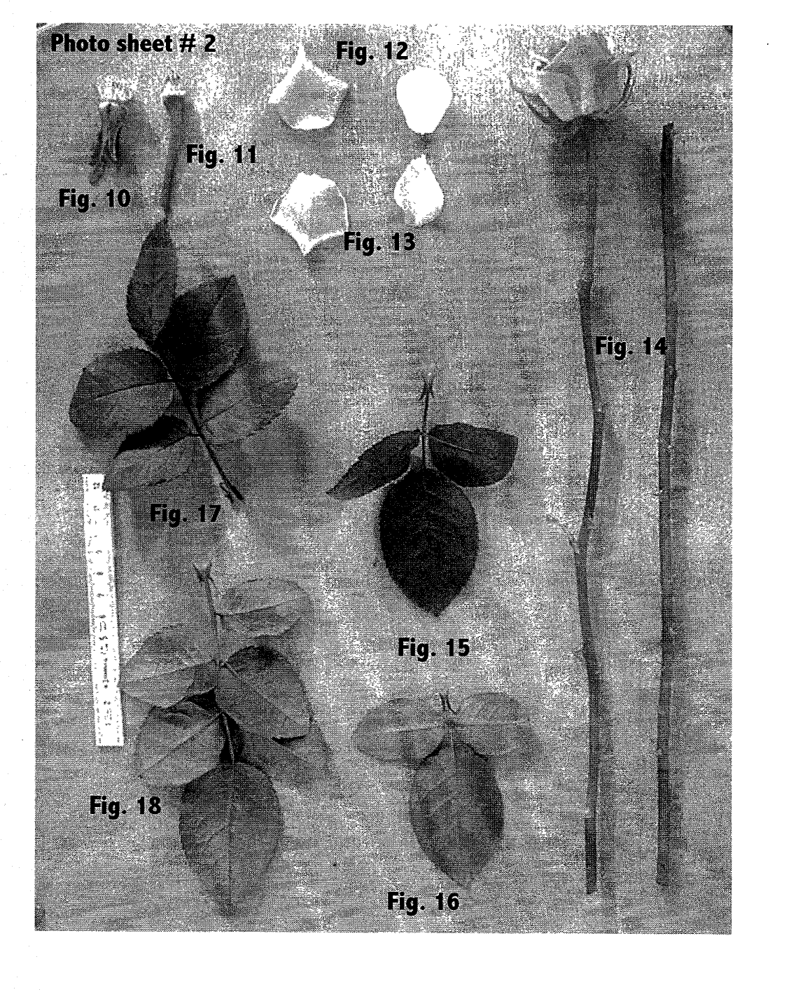

### DESCRIPTION OF THE PLANT

The following is a detailed description of ‘KORpries’, as observed in its growth in greenhouses in Klein Offenseth-Sparrieshoop, Germany, and glasshouses in California. The age of the observed plant was 11 to 13 months after propagation, produced as a cut-rose plant. The cultivation at both locations was based on 18 to 26° C. temperatures. Color references are made using The Royal Horticultural Society (London, England) Colour Chart, 1995, except where common terms of color are used. For a comparison, the nearest existing rose variety is ‘JACdeep’, U.S. Plant Pat. No. 9,344. Table 1 details several physical characteristics of ‘KORpries’ and ‘JACdeep’.

TABLE 1

| | KORpries | JACdeep |
|---|---|---|
| Petal color, upper surface | 62A | 46D |
| Petal color, reverse surface | 62A | 46D |
| Petal count | 30-35 | 30-35 |

### THE PLANT

Classification:

Botanical.—Rosa hybrida.

Commercial.—Hybrid tea rose.

Growth habit.—Very vigorous. Grows strong upright to bushy. Production time for floral stems is generally 6 to 8 weeks depending on average temperature, light level, and cultural practices.

Height.—Plants which were pruned at a height of 0.5 meter, produced floral stems 50 to 60 cm long.

Stem:

Color.—Young wood: 146B. Older wood: 137A.

Thorns.—Incidence: High number of thorns. Size: 4-7 mm. Color: 152C at base, with intonations of 153C at tip. Shape: Deep concave.

Surface.—Young wood: Smooth. Older wood: Smooth.

Stem diameter.—4-5 mm.

Internode length.—50-70 mm.

Numbers of internodes.—10-11.

### FOLIAGE

Arrangement: Alternate, compound with 3-7 leaflets per leaf, generally symmetrical, abundant, and flat in aspect. Stipules at petiole base.

Quantity of leaves: 10-11 per lateral branch.

Leaf size:

Length.—100-130 mm.

Width.—90-110 mm.

Petioles:

Color.—145C, with intonations of 187B.

Margins.—Have prickles of 187B.

Length.—3-20 mm.

Diameter.—About 1-2 mm.

Stipules:

Size.—10-15 mm.

Surface.—Smooth.

Color.—138A, with intonations of 183C.

Margin.—Serrated with stipitate glands.

Rachis:

Color.—138A, with intonations of 59B.

Margin.—Margin with stipitate glands, and prickles colored 187B.

Length.—30-80 mm.

Leaflets:

Edge.—Serrated.

Serration.—Single.

Shape.—Ovate with obtuse apex and obtuse base.

Texture.—Smooth.

Appearance.—Dull.

Size.—Length: 35-70 mm. Width: 25-50 mm.

Color.—Young foliage: Upper surface: 146B, with intonations of 181A. Lower surface: 146C with intonations of 181A. Mature foliage: Upper surface: 147A. Lower surface: 191B, with intonations of 181A.

### BUD

Size upon opening:

Length.—30-35 mm.

Width.—20-25 mm.

Shape: Cupped.

Color at one-quarter opening: 55C.

### FLOWERS

Flowering characteristics: Recurrent, generally 1 bud per flowering stem.

Duration: On the plant, flowers last from 12 to 15 days. When cut and placed in a vase, flowers last from 10 to 11 days.

Fragrance: None.

Size: 60-80 mm in diameter.

Shape of flower when viewed from the side:

Upon opening.—Cupped.

Open flower.—Cupped.

Peduncle:

Color.—Between 146B and 146C.

Texture.—Smooth.

Length.—50-70 mm.

Angle.—Upright.

Receptacle:

Surface.—Smooth, glabrous.

Shape.—Funnel-shaped.

Size.—Height: 5-8 mm. Width: 6-7 mm.

Color.—Between 146B and 146C.

Sepals:

Quantity.—5.

Size.—30-50 mm.

Shape.—Narrowly ovate with acute tip.

Texture.—Leathery.

Margin.—Foliaceous appendages on 4 of the 5 sepals.

Appearance.—Dull.

Color.—Upper surface: 146C, with intonations of 181A. Reverse surface: 146C, with intonations of 181A.

Petals:

Color.—Upon opening: Upper surface: 62A. Reverse surface: 62A. After opening: Upper surface: 62D. Reverse surface: 62D.

General tonality of open flower.—55C on the third day, 62D afterwards.

Basal petal spots.—Size: 8-10 mm. Color: 155A.

Reflex.—Outermost petals reflex backwards at opening. When fully open all petals reflex backwards. Texture: Smooth. Petal edge: Uniform. Quantity: Approximately 30-35 on average per flower.

Petal size.—Length: 50 mm. Width: 40 mm.

Shape.—Outer petals: Round to ovate. Inner petals: Ovate.

Petaloids.—Commonly none.

### REPRODUCTIVE ORGANS

Stamens: Approximately 40-45 on average per flower.

Pollen.—Color: 163A. Amount: Average.

Anthers.—Size: 2-3 mm. Color: 163A. Shape: Oblong.

Filaments.—Size: 5-7 mm. Color: 155B.

Pistils.—Approximately 30-35 on average per flower.

Stigmas.—Location: Below anthers. Color: 155B.

Styles.—Color: 57A. Length: 3-4 mm.

Aptitude to bear fruit: Poor.

Resistance to diseases: Above average resistance to mildew and Botrytis under normal growing conditions in Klein Offenseth-Sparrieshoop, Germany and California.

Productivity: Commonly produces approximately 210 to 220 flowers/m²·year under greenhouse conditions.

Hips/seeds: Unknown, the plant has not been grown to the stage of seed development due to the fact that the variety is developed for use as a flowering cut-rose only.

Winter hardiness and drought/heat tolerance: Due to the fact that this variety is a flowering cut rose plant developed for greenhouse cultivation only, the plant has not been tested for winter hardiness or drought/heat tolerance.

I claim:

1. The new and distinct variety of hybrid tea rose plant named ‘KORpries’ as described and illustrated.
